(19) United States
(12) Patent Application Publication
(10) Pub. No.: US 2003/0028157 A1
(43) Pub. Date: Feb. 6, 2003

(54) Support device with integrated pressure adjustment device and method of use

(76) Inventors: Joel T. Jusiak, Holland, NY (US); Roland E. Flick, Elma, NY (US)

Correspondence Address: Georgia Evans, NIXON PEABODY LLP, Clinton Square, P.O. Box 31051, Rochester, NY, 14603-1051, US

(21) Appl. No.: 10/195,654

(22) Filed: Jul. 12, 2002

Related U.S. Application Data
(60) Provisional application No. 60/305,237, filed on Jul. 13, 2001.

(51) Int. Cl. A61M 1/00

(52) U.S. Cl. 604/313; 604/315

(57) ABSTRACT

A protective and pressure normalizing device including a support member having an exterior wall and an interior wall which define a first chamber and a second chamber, and a pressure adjustment device sealed within at least a portion of the second chamber, the pressure adjustment device defining a third chamber and a medium transfer passageway interconnecting the first chamber and third chamber.

The present invention claims the benefit of U.S. Provisional Patent Application Serial No. 60/305,237, filed Jul. 13, 2001, which is hereby incorporated by reference in its entirety.

### FIELD OF THE INVENTION

The present invention generally relates to devices and methods for stabilizing and supporting a structure, especially a body part of a user. The invention, more specifically, is an inflatable device that provides protection for, and relieves pressure on the body part, especially when the body is in a supine or semi-Fowler's position

### BACKGROUND OF THE INVENTION

Devices for supporting a body extremity have been developed. For example, U.S. Pat. No. 5,489,259 to Jacobs et al. (“Jacobs”) relates to a pressure-normalizing single-chambered static pressure device for supporting and protecting a body extremity, in particular a heel. That device has an inflatable member with exterior and interior surfaces, a plurality of seams to connect the exterior and interior surfaces together along a line corresponding to the Achilles' tendon and the ankle bones, a plurality of apertures along those seams, and a nozzle.

In order to inflate the Jacobs' device, an external pump is attached to the nozzle and the device is inflated to a predetermined pressure by a third party or the patient, then the device is left alone and is said to conform to the extremity.

A problem with devices, such as those described in Jacobs, is that the device may be over-inflated or even under-inflated. Such results may become deleterious to the patient. In addition, when the devices are inflated with air, there is a significant risk of cross-contamination caused by different individuals orally adjusting the pressure of the medium through the nozzle. Moreover, an external device, such as a pump, is required to adjust the pressure of the medium within the device, even after the device is initially inflated to a desired level, to make modifications for climatic conditions, changes in elevation, or leakage.

### SUMMARY OF THE INVENTION

The present invention relates to a protective and pressure normalizing device. The device includes a support member having an exterior wall and an interior wall which define a first chamber and a second chamber, and a pressure adjustment device sealed within at least a portion of the second chamber, the pressure adjustment device defining a third chamber and a medium transfer passageway interconnecting the first chamber and third chamber.

The present invention also relates to a protective and pressure normalizing device for a body extremity. The device includes a support member having an exterior wall and an interior wall which define a first chamber, a second chamber, and a joint aperture. A manually actuated pump is sealed within at least a portion of the second chamber. The manually actuated pump includes a resilient member encapsulated within an envelope, wherein a portion of the envelope forms a one-way check valve in communication with the first chamber.

Another aspect of the present invention relates to a method for protecting and normalizing the pressure on a structure. The method involves providing a support member having an exterior wall and an interior wall which define a first chamber and a second chamber, and a pressure adjustment device sealed within at least a portion of the second chamber, the pressure adjustment device defining a third chamber and a medium transfer passageway interconnecting the first chamber and third chamber. The structure is positioned in the support member.

In the device and method of the present invention, the pressure of a medium within the device is adjusted through an integrated pressure adjustment device. As used herein, an integrated pressure adjustment device is located and sealed within the chamber or bladder being inflated. As further used herein, a pressure adjustment device is sealed within a chamber when it is in communication for the transfer of medium to the chamber whose pressure it is being used to adjust and is not in constant communication with a medium source external to the chamber. By sealing the pressure adjustment device within the chamber being inflated, thereby controlling and restricting the flow of medium to any location other than the chamber being inflated, the pressure adjustment device is unable to leak to the outside of the support member. Accordingly, the device more efficiently maintains its pressure. Further, the need for multiple components, such as the support device in combination with external pressure adjustment devices to adjust the pressure of the medium is eliminated. Thus, pressure of the medium within the chamber can be easily adjusted on-site by the user without the need for obtaining an external pressure adjustment device.

The device of the present invention, in a specific embodiment, is an inflatable device that provides protection for, and relieves pressure on the heel area of the foot, when the body is in a supine or semi-Fowler's position. When used to stabilize a foot, the device and method of the present invention can be used in order to prevent shortening of the Achilles tendon, i.e., “foot drop.” The device is adaptable to fit different sized body parts and can be worn on either a left or right extremity of the user. In addition, the device of the present invention minimizes pressure contact between the body part and any surface on which it is placed, for example, a hospital bed, thereby, decreasing the risk of the formation of decubitus ulcers on the body part as a result of such contact (while keeping the device lightweight). Moreover, the device of the present invention limits contact between the body part of a user, especially the toe area of the foot, and objects in the user's surroundings such as sheets, blankets, etc. Further, when used for the foot, the device separates the ankles, preventing them from crossing and thereby reducing additional pressure when the hips are rotated. In addition, the device allows for air circulation and provides protection for the sides and bottom of the foot or any other body part positioned in the device.

### BRIEF DESCRIPTION OF THE DRAWINGS

FIG. 1 is a perspective, partially cutaway view of a first embodiment of the device of the present invention including a pressure adjustment device;

FIG. 2 is a top view of the pressure adjustment device of FIG. 1;

FIG. 3 is a partial, cross-sectional view of the device of FIG. 1 taken along the lines 3-3;

FIG. 4 is a perspective view of FIG. 1 showing edges 72 and 74 which are joined to form the device of FIG. 1;

FIG. 5 is a cross-sectional view of FIG. 4 taken along the lines 5-5;

FIG. 6 is an another embodiment of FIG. 5;

FIG. 7 is an another embodiment of FIG. 5;

FIG. 8 is an another embodiment of FIG. 5; and

FIG. 9 is a side perspective view of an alternative embodiment of FIG. 1.

### DETAILED DESCRIPTION OF THE INVENTION

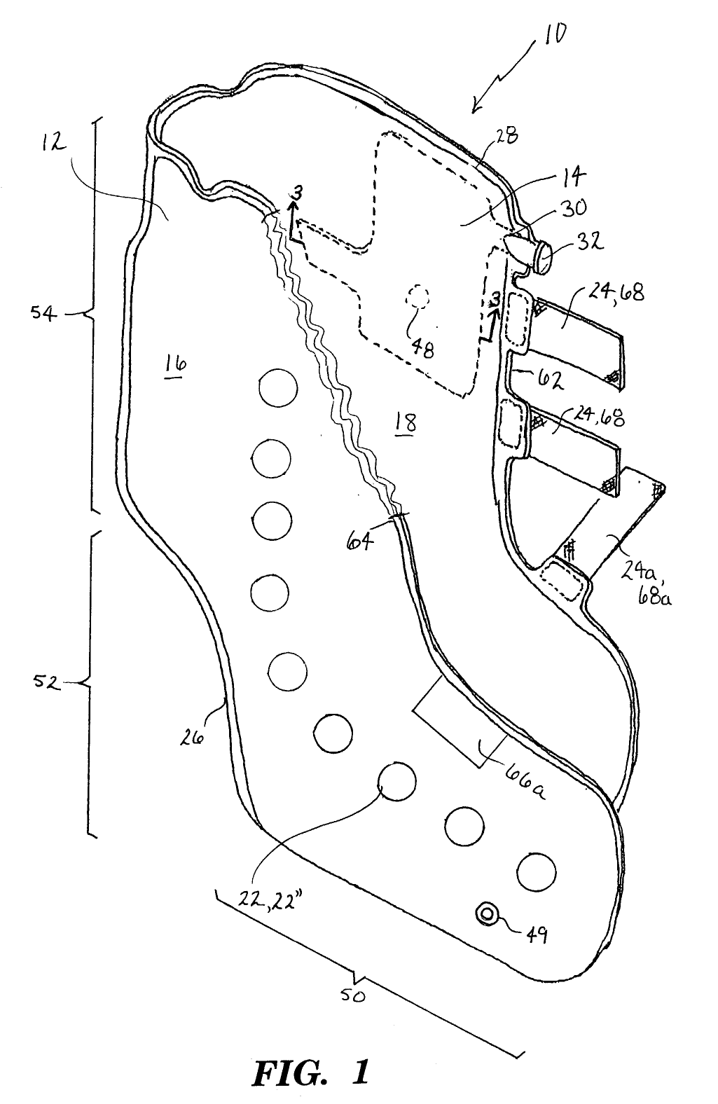

Referring to FIGS. 1-4, the present invention relates to a device 10 including a support member 12 which includes an integrated pressure adjustment device 14 within the support member 12. In particular, the support member has an exterior wall 16 and an interior wall 18 which define a first chamber 20, shown in FIGS. 3 and 5-8. The pressure adjustment device 14 is located within the chamber 20 whose pressure it is adjusting and can be located at any position within the chamber. The support member 12 can be used to stabilize a body part (e.g., a body extremity) or any other structure. When used to stabilize a foot, the device and method of the present invention can be used in order to prevent shortening of the Achilles tendon, i.e., “foot drop.” In addition, the device of the present invention minimizes pressure contact between the body part and any surface on which it is placed as well as other objects in the user's surrounding, thereby decreasing the risk of the formation of decubitus ulcers on the body part as a result of such contact.

FIG. 1 shows a perspective view of a device 10 according to the present invention. Device 10 includes a support member 12 which has an exterior wall 16 and an interior wall 18 which define a first chamber 20, shown in FIGS. 3 and 5-8. The support member 12 also includes a plurality of non-support regions 22, one or more securing devices 24, and an aperture for a joint of a user 26. In addition, the support member 12 includes a pressure adjustment device 14.

Referring back to FIG. 1, in this embodiment, the pressure adjustment device 14 is positioned and sealed within the first chamber 20 at a first end 28 adjacent a top edge of the support member 12. However, the pressure adjustment device 14 may be any suitable size or shape and may be positioned at any location within the first chamber 20.

As shown in FIGS. 1-3, in this particular embodiment, the pressure adjustment device 14 is a manually actuated pump which can be used to adjust the pressure of a medium in the first chamber 20. The pressure adjustment device 14 is sealed and encapsulated within the first chamber 20 by sealing the exterior wall 16 to the interior wall 18 around the pressure adjustment device 14 by heat-sealing, radio-frequency sealing, or any other suitable technique, thereby forming a second chamber or subchamber 20a. The second chamber 20a includes an inlet opening 30 which provides access to a source of a medium, such as air, used to fill chambers 20 and 20a, but which is closed with plug 32 except when medium is to be added to the chambers 20, 20a.

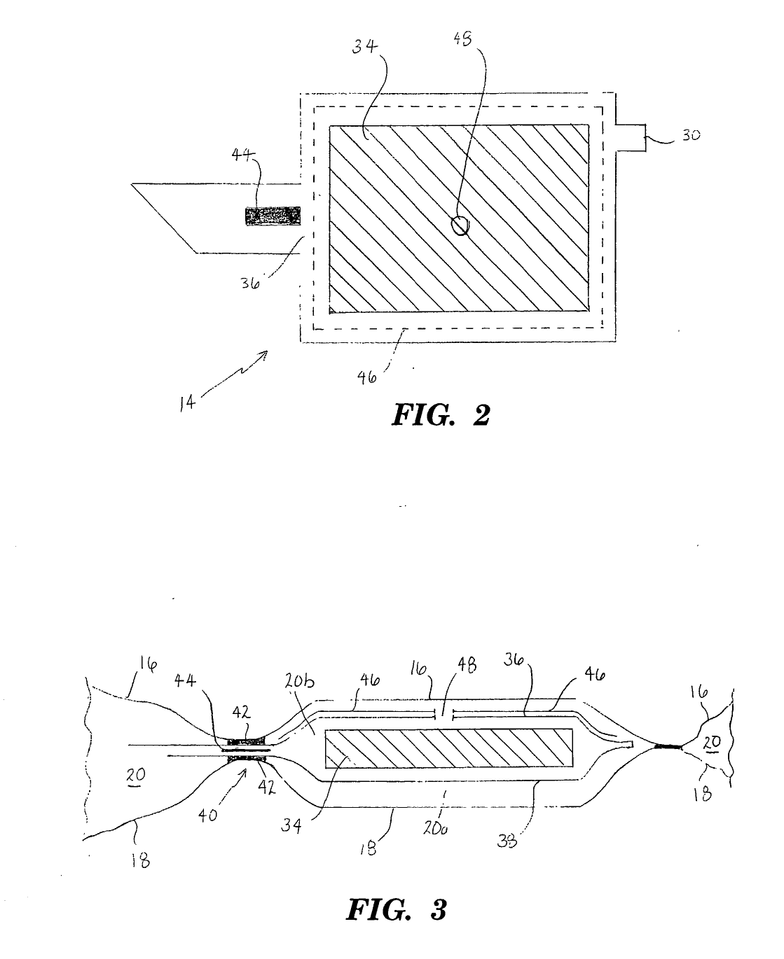

More specifically, as shown in FIGS. 2-3, the pump includes a piece of foam 34 encapsulated in an envelope formed by a first sheet 36 and a second sheet 38 positioned in the second chamber 20a. Although two sheets 36 and 38 are shown, the foam 34 may be encapsulated in a single sheet. The envelope formed by first and second sheets 36, 38 defines a third chamber 20b within second chamber 20a. The foam 34 is an open-cell, resiliently flexible material, such as polyurethane foam. However, any resilient member may be used, such as springs and plastic molded parts. In this embodiment, the first and second sheets 36, 38 are plastic sheets which are from about 0.005 inches to about 0.020 inches in thickness. However, the first and second sheets 36, 38 may be made of any material in any desired thickness and may be the same material or a different material than the exterior wall 16 and interior wall 18. The first sheet 36 and second sheet 38 form a “duck-bill” type check valve 40 at seal 42. Such valves are known in the art and are described, for example, in U.S. Pat. No. 5,144,708, which is hereby incorporated by reference in its entirety. Briefly, the first sheet 36 and second sheet 38 are adhered at seal 42 to the exterior wall 16 and interior wall 18 of the support member 12. A barrier material 44 is disposed on the inner surface of one or both of the first and second sheets 36, 38 through the seal 42 to prevent fusing together of the first and second sheets 36, 38 at that area of the seal 42. The barrier material 44 defines a central channel through the seal 42 which allows one-way fluid flow from the third chamber 20b to the first chamber 20.

Referring to FIG. 3, in this particular embodiment, the pump also includes a non-adhesive layer 46 adjacent the first sheet 36, although the non-adhesive layer 46 may also be adjacent the second sheet 38. The non-adhesive layer 46 prevents the first sheet 36 and/or the second sheet 38 from adhering to inner surfaces of the exterior wall 16 and interior wall 18, and may be any suitable material, such as synthetic woven material or synthetic non-woven material. However, adhesion to the inner surfaces of the exterior wall 16 and interior wall 18 may be prevented by treating either the inner surfaces of the exterior wall 18 and interior wall 18 and/or the outer surfaces of the first and second sheets 36, 38 to make them non-adhesive. For example, the inner surfaces of the exterior wall 18 and interior wall 18 and/or the outer surfaces of the first and second sheets 36, 38 may be coated with a non-adhesive material, such as printers ink, thereby eliminating the need for a non-adhesive layer 46 in the pressure adjustment device 14.

In addition, in this embodiment, the pressure adjustment device 14 includes an intake 48 for drawing a medium, such as air, into the pressure adjustment device 14 and into the chambers 20a and 20b, and, ultimately, 20. Referring to FIG. 1, the support member 12 may also include a release valve 49 for releasing medium from first chamber 20.

Although a foam-based, manually actuated pump has been described, the pressure adjustment device 14 may be any suitable device. Other suitable pressure adjustment devices 14 include, but are not limited to, squeeze-ball type pumps, bellows, tire pumps, or electronic pumps.

As shown in FIG. 1, the support member 12 forms a single first chamber 20. However, the first chamber 20 of the support member 12 may include a series of interconnected cells that exchange the medium and redistribute weight of the structure placed within the device.

The support member 12 with non-support regions 22 has an exterior wall 16 and an interior wall 18. The exterior and interior walls 16, 18 can be constructed of the same or different materials.

The interior wall 18 is constructed of a lightweight cotton, gauze, paper, or synthetic material which may be woven or non-woven, or any other material that is breathable and suitable for use in a hospital, alternative care, or home care environment, when the device is used to support a body part. The interior wall 18 is strong enough to secure a body part of a user and capable of being comfortably placed in contact with the body part of a user. In use, at least a portion of the interior wall 18 of the support member 12 contacts at least a portion the body part of the user.

The exterior wall 16 can be constructed of any type of material so long as it meets some minimal characteristics. These minimal characteristics, which apply to both materials 16 and 18, when the device is used to support a body part, include not applying excessive shear force to a user's tissue, providing adequate wicking characteristics so that moisture is drawn away from the skin of the body part to keep the body part at a normal temperature, and providing sufficient flexibility and pliability to conform readily to the shape of the user's body part. The exterior wall 16 and interior wall 18 may be constructed to be disposable or reusable.

In an alternative embodiment, the exterior wall 16 may include an exterior reinforcing material layer and the interior wall may include an interior wicking material layer to cool and wick moisture. The exterior reinforcing material layer and interior wicking material layer may be made of the same or different materials than the exterior wall 16 and interior wall 18. In this embodiment, at least a portion of the interior wicking material layer contacts at least a portion of the body part of the user.

Referring to FIG. 1, the device 10 is divided into at least three distinct components 50, 52, and 54 that correspond to a body extremity having a joint (elbow, knee, or heel/ankle) of a user. However, the device 10 may be divided into any number of components. The body extremity is divided into a posterior, anterior, and the two other sides.

As shown in FIG. 1, the components are a lower portion 50, an intermediate portion 52, and an upper portion 54. The interior wall 18 of the lower portion 50 contacts at least parts of the anterior and other sides of the foot (i.e., a lower region of the body extremity). The interior wall 18 of the intermediate portion 52 contacts at least a portion of the other sides of the body extremity that corresponds to the heel, which is on the posterior side of the body extremity (i.e., an intermediate region of the body extremity) and includes the joint aperture 26. The interior wall 18 of the upper portion 54 contacts at least a portion of the posterior and other sides of the calf of the body extremity (i.e., an upper portion of the body extremity). Although the device 10 shown in FIG. 1 relates to the ankle, heel, and foot of a user, the device may be used with other body parts, e.g., a knee or an elbow, and the interior wall 18 would contact surfaces of the body part other than that described above.

The joint aperture 26 exposes the joint of the body part. In this particular example, the joint is the heel. However, the joint could be any suitable joint, such as the elbow or knee. Although this particular embodiment includes a joint aperture 26, the device could be provided without a joint aperture.

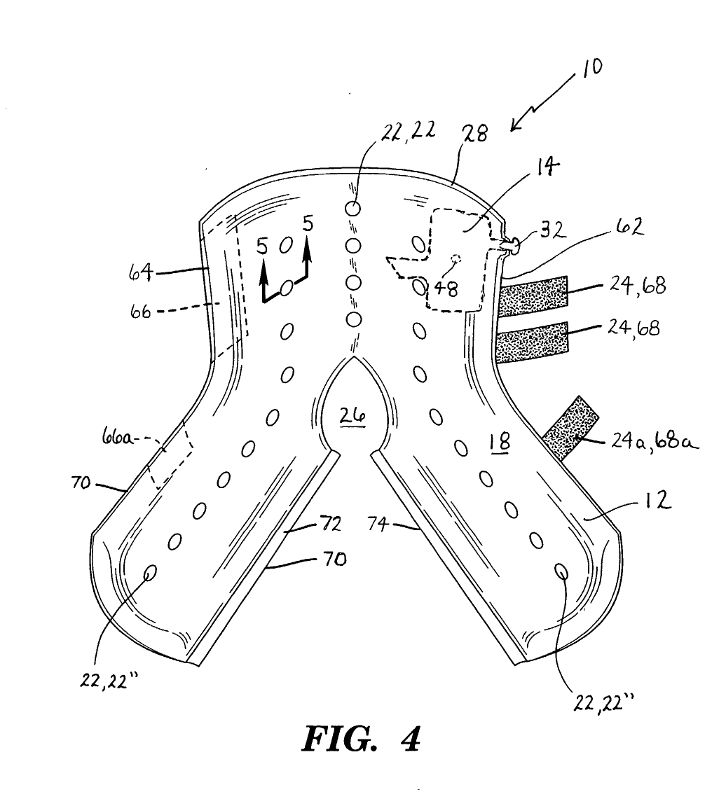

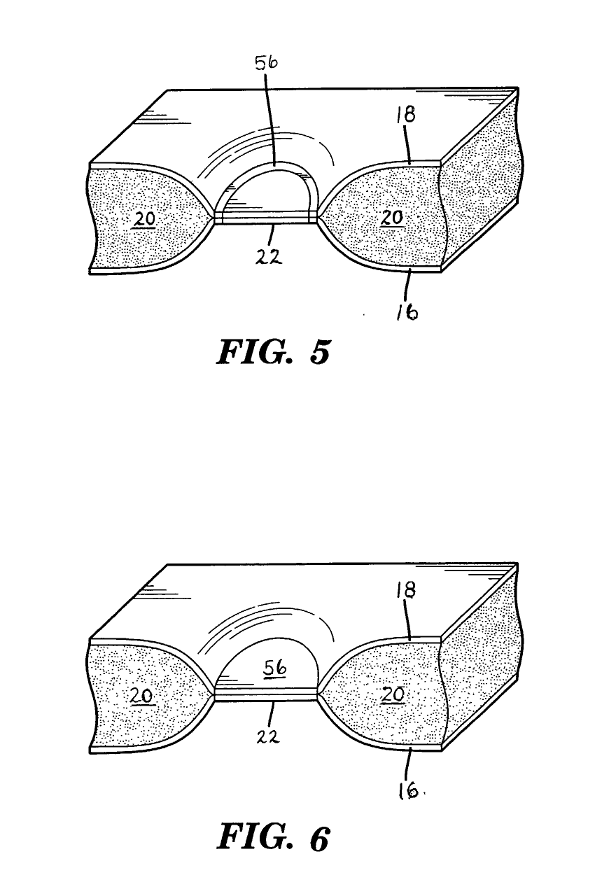

To relieve pressure on the body part, the support member 12 has a plurality of non-support regions 22 interspaced throughout it. In one embodiment, shown in FIG. 4, there is a first set of non-support regions 22′ positioned along a line corresponding to an Achilles' tendon of the calf. A second set of the nonsupport regions 22″ is positioned along a line corresponding to the ankle bone of the body extremity. Each of non-support regions 22′, 22″ is interspaced from the others, as shown in FIG. 4, and highlighted in FIG. 5—a cross-section of FIG. 4 taken along the lines 5-5.

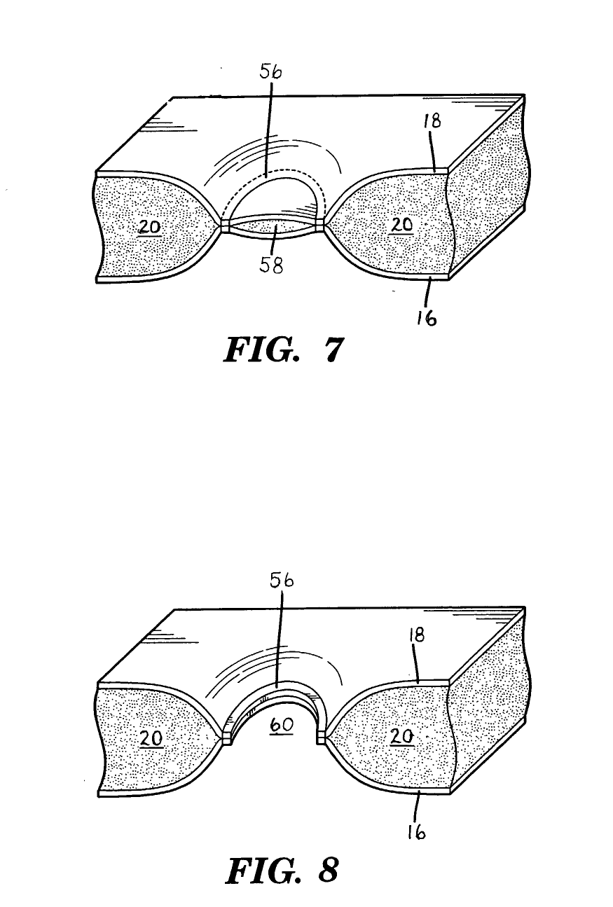

As shown in FIG. 5, the non-support region 22 is formed by bonding predetermined portions of the interior wall 18 to the exterior wall 16 at predetermined point 56. In some embodiments, the predetermined point 56 is a pre-selected area of the non-support region 22 wherein the remainder of the nonsupport region 22 is flat, as shown in FIG. 5, or the predetermined point 56 is the entire non-support region 22 as shown in FIG. 6 (an alternative embodiment of FIG. 5), or the predetermined point 56 is a pre-selected area of the non-support region 22 wherein the remainder of the region 22 is filled with a fluid or a predetermined piece of material 58, as shown in FIG. 7 (an alternative embodiment of FIG. 5), or the predetermined point 56 is a pre-selected area of the non-support region 22 wherein within a predetermined portion of the remainder of the region 22 is an aperture 60, as shown in FIG. 8 (an alternative embodiment of FIG. 5). The predetermined point 56 can be made by a heat-seal, ultrasonic sound, or other conventional means.

Referring to FIGS. 3 and 5-8, the exterior wall 16 and interior wall 18 of the support member 12 form a first chamber 20 which includes a medium having an initial predetermined pressure. For example, the first chamber 20 of the support member 12 is filled with a medium to a pressure of about 0-50 mmHg. In one embodiment, the chamber 20 is filled with a medium to a pressure of about 0-15 mmHg. Such pressures minimize stretch of the interior and exterior walls 16, 18 of the support member 12 due, for example, to elevation or temperature changes. However, any desired pressure can be used. In addition, any suitable medium can be used. The medium is typically a fluid, such as gases (e.g., air) or liquids (e.g., water), suspensions, gelastic materials, as sold by EdiZone, Inc. of Utah, or any other material that reduces pressure on a body part. In this particular embodiment, the medium is air.

The pressure of the air within the chamber 20 is adjusted by activating the integrated pressure adjustment device 14. In particular, the pressure of the air is adjusted by removing the plug 32 from inlet opening 30 and manually operating the pressure adjustment device 14 to pull air into the chambers 20, 20a, and 20b from ambient. However, when using a medium other than air, the inlet opening 30 is connected to a source of medium. By adjusting the pressure of the medium through the integrated pressure adjustment device 14, the need for external devices to adjust the pressure of the medium is eliminated. In addition, leakage from the integrated pressure adjustment device 14 is contained within the support member 12 by closing the opening 30 with plug 32, except when adding medium to the device 10. This allows the pressure within the support member 12 to be easily maintained.

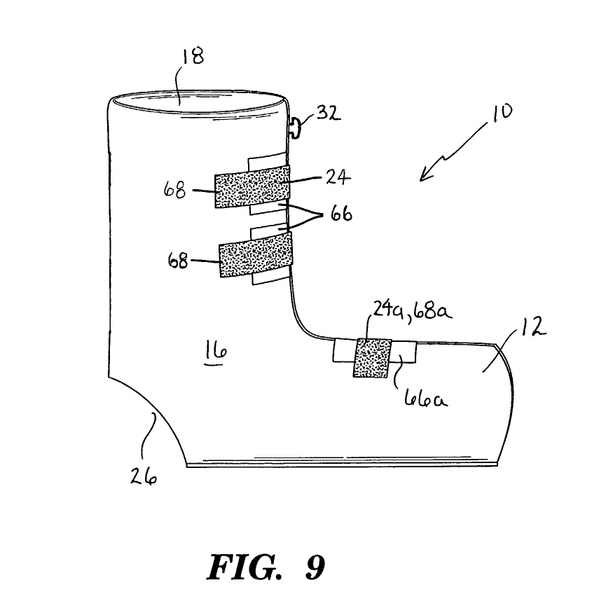

FIG. 9 illustrates an alternative embodiment of the device of FIG. 1 without any non-support members 22.

Referring to FIGS. 1, 4, and 9, the present invention includes a plurality of securing devices 24. Each securing device 24 may be a support surface or a non-support surface. In either embodiment, each securing device 24 secures first and second edges 62, 64 of support member 12 to within a desired distance of each other and prevents device 10 from disengaging with the body part. Each securing device 24 is divided into two units, a receiver 66 and an extender 68. In the embodiment shown in FIGS. 1 and 4, the extender 68 protrudes from edge 62, however, the extender 68 may protrude from either edge. Receiver 66 receives the extender 68 and secures the extender 68 into a desired position. In the present embodiments, the receiver 66 is a loop device, and the extender 68 has a hook device that connects with the loop device, although any type of securing device may be used. The receiver 66 can be a single unit, as illustrated in FIG. 4, or separate units for each extender 68, as illustrated in FIG. 9. In addition, the device may include one or a plurality of securing devices 24. To attain maximum engagement between the edges 62 and 64, each securing device 24 in each embodiment has a particular position such that receiver 66 receives extender 68 at a 90° angle.

In another embodiment, as shown in FIGS. 1, 4, and 9, an alternative securing device 24a is positioned on the lower portion 50 of the device 10. This alternative securing device 24a further secures the device 10 to the body part and includes a receiver 66a and an extender 68a. However, the device 10 may be provided without securing device 24a.

The device 10 illustrated in FIG. 4 is in a pre-assembled position. The device 10 is put together by joining the exterior wall 16 to the interior wall 18 at an edge 70. Once the edge 70 is sealed, normally by heat sealing, the two particular edges, 72 and 74, are further joined together to form the device 10.

In accordance with one embodiment of the present invention, the device 10 is pre-filled with a medium. By pre-filling the device 10, the user is less likely to cause damage to its body part. Many individuals ignore the fact that when the temperature and/or air pressure of the environment is altered the pressure within the device 10 is also altered. Therefore, by pre-filling the device 10, the user is unable to over inflate or under inflate the device 10. The user can then adjust the pressure of the medium within the chamber, for example, to make up for volume changes due to climatic conditions and changes in elevation.

The device 10 of the present invention can be used to stabilize a body part. For example, the support member 12 can be used for stabilizing an ankle while relieving pressure on the heel of the foot, a knee while relieving pressure on the knee of the leg, or an elbow while relieving pressure on the elbow of the arm. The knee, ankle/heel, and elbow are collectively referred to as a flexible joint or a bendable joint.

In use, the user positions a body part, such as a leg, foot, or arm, in the support member 12. If present, securing devices 24 and 24a are used to secure the support member 12 to the body part. The user then fills/adjusts the chamber 20 of the support member to a desired pressure using the pressure adjustment device 14. In particular, to increase the pressure within the chamber 20, the user removes plug 32, positions a finger or thumb over intake 48, and alternately depresses and releases finger pressure on the foam 34 for a sufficient number of pulsations to reach the desired pressure. The exterior and interior walls 16, 18 may be transparent in order to allow the user to position a finger or thumb over intake 48 or a marking may be provided on the exterior and/or interior walls 16, 18 to allow the user to correctly position his/her finger or thumb. The user then repositions plug 32 in opening 30 to seal the pressure adjustment device within device 10. To decrease the pressure within the chamber 20, the user opens a release valve 49, if present, on the support member 12, which allows medium within the chamber 20 to be released. Alternatively, the user may fill/adjust the chamber 20 to a desired pressure prior to securing the support member 12 to the body part.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, the recited order of processing elements or sequences, or the use of numbers, letters, or other designations therefor, is not intended to limit the claimed process to any order except as may be specified in the claims.

What is claimed is:

1. A protective and pressure normalizing device comprising:
a support member having an exterior wall and an interior wall which define a first chamber and a second chamber, and
a pressure adjustment device sealed within at least a portion of the second chamber, the pressure adjustment device defining a third chamber and a medium transfer passageway interconnecting the first chamber and third chamber.

2. The device according to claim 1 wherein the pressure adjustment device comprises a pump.

3. The device according to claim 2 wherein the pump is a manually actuated pump comprising a resilient member encapsulated within the third chamber and wherein the fluid passageway comprises a one-way check valve.

4. The device according to claim 3 wherein the resilient member is a foam member.

5. The device according to claim 3 wherein the pump further comprises an intake which extends between the second and third chambers.

6. The device according to claim 1 wherein the second chamber comprises an inlet opening adjustable between an open position and a closed position and wherein the inlet opening is connected to a medium source when in the open position.

7. The device according to claim 1 wherein the support member further comprises a release valve.

8. The device according to claim 1 wherein the support member further comprises a joint aperture.

9. The device according to claim 1 further comprising a wicking material layer adjacent the interior wall.

10. The device according to claim 1 further comprising a reinforcing material layer adjacent the exterior wall.

11. The device according to claim 1 wherein the first chamber is filled with a medium to an internal pressure of about 0 mmHg to about 50 mmHg.

12. The device according to claim 11 wherein the medium is air.

13. The device according to claim 1 wherein the first chamber comprises a single chamber.

14. The device according to claim 1 further comprising a plurality of interspaced non-support members in the support member.

15. The device according to claim 1 wherein the support member comprises:
a lower portion, wherein at least a portion of the interior wall of the lower portion contacts at least a portion of a lower region of a body part;
an intermediate portion, wherein at least a portion of the interior wall of the intermediate portion contacts at least a portion of an intermediate region of the body part; and
an upper portion, wherein at least a portion of the interior wall of the upper portion contacts at least a portion of an upper region of the body part.

16. The device according to claim 1 further comprising one or more securing devices, wherein the one or more securing devices attach at least a portion of a first edge of the support member to at least a portion of a second edge of the support member.

17. A protective and pressure normalizing device for a body extremity comprising:
a support member having an exterior wall and an interior wall which define a first chamber, a second chamber, and a joint aperture, and
a manually actuated pump sealed within at least a portion of the second chamber, wherein the manually actuated pump comprises a resilient member encapsulated within an envelope and wherein a portion of the envelope forms a one-way check valve in communication with the first chamber.

18. The device according to claim 17 wherein the pump further comprises an intake which extends between the second chamber and the envelope.

19. The device according to claim 17 wherein the second chamber comprises an inlet opening adjustable between an open position and a closed position and wherein the inlet opening is connected to a medium source when in the open position.

20. The device according to claim 17 wherein the support member further comprises a release valve.

21. The device according to claim 17 wherein the resilient member is a foam member.

22. The device according to claim 17 further comprising a wicking material layer adjacent the interior wall.

23. The device according to claim 17 further comprising a reinforcing material layer adjacent the exterior wall.

24. The device according to claim 17 wherein the first chamber is filled with a medium to an internal pressure of about 0 mmHg to about 50 mmHg.

25. The device according to claim 24 wherein the medium is air.

26. The device according to claim 17 wherein the first chamber comprises a single chamber.

27. The device according to claim 17 further comprising a plurality of interspaced non-support members in the support member.

28. The device according to claim 17 wherein the support member comprises:
a lower portion, wherein at least a portion of the interior wall of the lower portion contacts at least a portion of a lower region of a body part;
an intermediate portion, wherein at least a portion of the interior wall of the intermediate portion contacts at least a portion of an intermediate region of the body part; and
an upper portion, wherein at least a portion of the interior wall of the upper portion contacts at least a portion of an upper region of the body part.

29. The device according to claim 17 further comprising one or more securing devices, wherein the one or more securing devices attach at least a portion of a first edge of the support member to at least a portion of a second edge of the support member.

30. A method for protecting and normalizing the pressure on a structure comprising:
providing a support member having an exterior wall and an interior wall which define a first chamber and a second chamber and a pressure adjustment device sealed within at least a portion of the second chamber, the pressure adjustment device defining a third chamber and a medium transfer passageway interconnecting the first chamber and third chamber, and
positioning the structure in the support member.

31. The method according to claim 30 further comprising adjusting a pressure of a medium within the first chamber with the pressure adjustment device.

32. The method according to claim 31 wherein the adjusting comprises adjusting the pressure of the medium to about 0 mmHg to about 50 mmHg.

33. The method according to claim 31 wherein the medium is air.

34. The method according to claim 30 wherein the pressure adjustment device comprises a pump.

35. The method according to claim 34 wherein the pump is a manually actuated pump comprising a resilient member encapsulated within the third chamber and wherein the medium transfer passageway comprises a one-way check valve.

36. The method according to claim 35 wherein the resilient member is a foam member.

37. The method according to claim 34 wherein the pump further comprises an intake which extends between the second and third chambers.

38. The method according to claim 30 wherein the second chamber comprises an inlet opening adjustable between an open position and a closed position and wherein the inlet opening is connected to a medium source when in the open position.

39. The method according to claim 30 wherein the support member further comprises a release valve.

40. The method according to claim 30 wherein the support member further comprises a wicking material layer adjacent the interior wall.

41. The method according to claim 30 wherein the support member further comprises a reinforcing material layer adjacent the exterior wall.

42. The method according to claim 30 wherein the first chamber comprises a single chamber.

43. The method according to claim 30 wherein the support member further comprises a plurality of interspaced non-support members in the support member.

44. The method according to claim 30 wherein the support member comprises:
a lower portion, wherein at least a portion of the interior wall of the lower portion contacts at least a portion of a lower region of a body part;
an intermediate portion, wherein at least a portion of the interior wall of the intermediate portion contacts at least a portion of an intermediate region of the body part; and
an upper portion, wherein at least a portion of the interior wall of the upper portion contacts at least a portion of an upper region of the body part.

45. The method according to claim 30 wherein the support member further comprises a plurality of securing devices, wherein the securing devices attach at least a portion of a first edge of the support member to at least a portion of a second edge of the support member.
